(19) United States
(12) Patent Application Publication
(10) Pub. No.: US 2017/0000471 A1
(43) Pub. Date: Jan. 5, 2017

(54) Tissue Displacement Apparatus For Medical Procedures

(71) Applicant: MAYO FOUNDATION FOR MEDICAL EDUCATION AND RESEARCH, Rochester, MN (US)

(72) Inventors: Matthew R. Callstrom, Rochester, MN (US); J. William Charboneau, Rochester, MN (US); David A. Woodrum, Kasson, MN (US)

(21) Appl. No.: 15/200,416

(22) Filed: Jul. 1, 2016

Related U.S. Application Data
(60) Provisional application No. 62/188,219, filed on Jul. 2, 2015.

(51) Int. Cl. A61B 17/02

(52) U.S. Cl. CPC A61B 17/0206; A61B 2017/00101

(57) ABSTRACT

A tissue displacement apparatus used during a tissue ablation procedure is provided. In some configurations, the tissue displacement apparatus is configured to displace tissue within a patient and thermally isolate the displaced tissue.

### CROSS-REFERENCES TO RELATED APPLICATIONS

The present disclosure is based on, claims priority to, and incorporates herein by reference in its entirety, U.S. Provisional Patent Application No. 62/188,219, filed Jul. 2, 2015, and entitled “Tissue Displacement Apparatus for Medical Procedures.”

### STATEMENT REGARDING FEDERALLY SPONSORED RESEARCH

Not Applicable.

### BACKGROUND

The present disclosure relates generally to systems and methods for performing medical procedures and, more specifically, to a tissue displacement apparatus that may be used, for example, during ablation procedures.

Tissue ablation is used to destroy or damage (ablate) aberrant tissue on or within a patient's body. Typically, an ablation probe is placed in contact with the aberrant tissue, which then ablates the aberrant tissue. The ablation probe exposes the aberrant tissue to either heat (e.g., radiofrequency ablation) capable of ablating the aberrant tissue, or cold (e.g., cryoablation) capable of ablating the aberrant tissue. The temperatures generated by the ablation probe are sufficiently hot or cold to damage healthy tissue adjacent to the aberrant tissue. Therefore, it is desirable to isolate or protect healthy tissue adjacent to or surrounding the aberrant tissue during the ablation procedure.

### BRIEF SUMMARY

The present disclosure provides a tissue displacement apparatus that can be used, for example, during a tissue ablation procedure or other medical procedures. In particular, the tissue displacement apparatus is capable of displacing tissue in a patient and thermally isolating the displaced tissue.

In one aspect, the present disclosure provides a tissue displacement apparatus configured to be slideably received within an external tube placed within a patient's body. The tissue displacement apparatus includes a tube defining a central lumen and a distal end. The tube is configured to be moveable between a first tube position where the distal end of the tube is arranged within the external tube and a second tube position where the distal end of the tube protrudes from the external tube. The tissue displacement apparatus further includes an expandable cage arranged on the distal end of the tube. The expandable cage is expandable between a first cage position where the expandable cage is collapsed and a second cage position where the expandable cage is expanded. When the tube is in the first tube position, the expandable cage is in the first cage position. When the tube moves to the second tube position, the expandable cage expands to the second cage position.

In another aspect, the present disclosure provides a tissue displacement apparatus configured to be received within an external tube placed within a patient's body. The tissue displacement apparatus including a tube defining a central lumen, a distal end, and a proximal end. The tube is configured to be moveable between a first tube position where the distal end of the tube is arranged within the external tube and a second tube position where the distal end of the tube protrudes from the external tube. The tissue displacement apparatus further includes an expandable cage arranged on the distal end of the tube and expandable between a first cage position where the expandable cage is collapsed and a second cage position where the expandable cage is expanded. The tissue displacement apparatus further includes an actuation element arranged on the proximal end of the tube. The actuation element includes a push button configured to expand the expandable cage between the first cage position and the second cage position when pressed.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF DRAWINGS

The present disclosure will be better understood and features, aspects and advantages other than those set forth above will become apparent when consideration is given to the following detailed description thereof. Such detailed description makes reference to the following drawings.

FIG. 1 shows a pictorial view of a tissue displacement apparatus in accordance with one aspect of the present disclosure.

FIG. 2 shows the tissue displacement apparatus of FIG. 1 with an expandable cage in a second cage position.

FIG. 3 shows a pictorial view of a tissue displacement apparatus in accordance with another aspect of the present disclosure.

FIG. 4 shows the tissue displacement apparatus of FIG. 3 with an expandable cage in a second cage position.

FIG. 5 shows a pictorial view of the tissue displacement apparatus of FIG. 1 including one or more anchors.

FIG. 6 shows a pictorial view of the tissue displacement apparatus of FIG. 1 including one or more temperature sensors.

FIG. 7 shows a pictorial view of a tissue displacement apparatus where an expandable cage defines an asymmetric shape in a second cage position in accordance with another aspect of the present disclosure.

FIG. 8 shows a pictorial view of a tissue displacement apparatus where an expandable cage defines a substantially round shape in a second cage position in accordance with yet another aspect of the present disclosure.

FIG. 9 shows a pictorial view of a tissue displacement apparatus where an expandable cage defines an differential shape in a second cage position in accordance with still another aspect of the present disclosure.

### DETAILED DESCRIPTION

Ablating aberrant tissue within a patient's body, for example ablating a tumor in a patient's liver, is an image guided procedure where an ablation probe is navigated through the patient's body. While navigating the ablation probe through the patients body, there is often healthy tissue adjacent to or in the path of the ablation probe. In order for the ablation probe to reach the aberrant tissue, the healthy tissue must be displaced to provide space for the ablation probe. Additionally, the healthy tissue surrounding or adjacent to the aberrant tissue must be protected from the extreme, and damaging, temperatures generated by the ablation probe.

Currently, a physician, or other trained medical professional, performing an ablation procedure within a patient's body could use their hand(s) to displace tissue in the path of the ablation probe and/or adjacent to the aberrant tissue. However, this method expends one, or both, of the physician's hands and the tissue displacement is limited by the access the physician has to the tissue in the path of the ablation probe and/or adjacent to the aberrant tissue. Alternatively, one or more balloons could be placed in desired locations within the patient and inflated to displace the tissue in the path of the ablation probe and/or adjacent to the aberrant tissue. However, balloons will shift and follow a path of least resistance when inflated within the patient's body, and balloons can be deflated due to the extreme temperatures generated by the ablation probe being communicated to the balloons.

Healthy tissue adjacent to or surrounding the aberrant tissue can currently be protected by providing a fluid, such carbon dioxide, to attempt to thermally isolate the healthy tissue. However, fluids, especially gases, have very low viscosities and can easily flow away from the healthy tissue under the force of gravity or other unbalancing forces.

Due to the current difficulties in displacing tissue in the path of the ablation probe or adjacent to the aberrant tissue and thermally isolating healthy tissue from the extreme, and damaging, temperatures generated by the ablation probe, it would be desirable to have a tissue displacement apparatus capable of selectively and precisely displacing tissue in the path of the ablation probe or adjacent to the aberrant tissue, and thermally isolating healthy tissue from the extreme temperatures generated by the ablation probe.

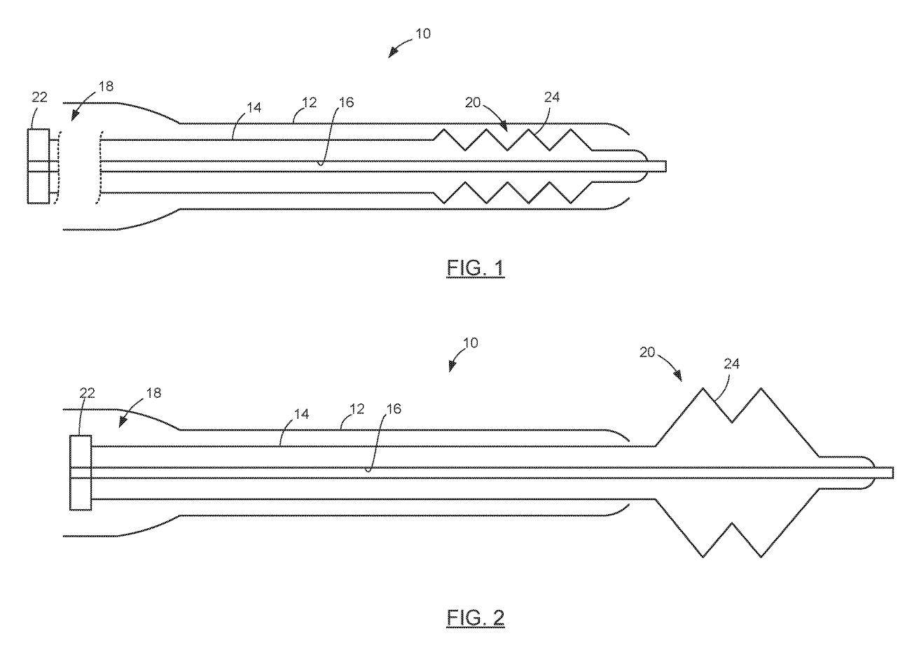

FIGS. 1 and 2 show one non-limiting example of a tissue displacement apparatus 10 in accordance with the present disclosure. The tissue displacement apparatus 10 is configured to be slideably received within an external tube 12 placed within a patient's body. The external tube 12 can be a trocar, or any other tube configured to be inserted into a patient's body. The tissue displacement apparatus 10 includes a tube 14 defining a central lumen 16 extending throughout the tube 14, a proximal end 18, and a distal end 20. The tube 14 is configured to be moveable within the external tube 12 between a first tube position (FIG. 1) where the distal end 20 of the tube 14 is arranged within the external tube 12 and a second tube position (FIG. 2) where the distal end 20 of the tube 14 protrudes from the external tube 12. The tube 14 can be fabricated from a low thermal conductivity, biocompatible material (e.g., a nickel titanium alloy (nitinol), stainless steel, or plastic).

In the illustrated non-limiting example, the proximal end 18 of the tube 14 includes an actuation element 22 in the form of a knob. The actuation element 22 provides a physician, or other trained medical professional, something to grip while moving the tube 14 between the first tube position (FIG. 1) and the second tube position (FIG. 2). In other non-limiting examples, the actuation element 22 may be in the form of any mechanical structure configured to provide a physician something to grip.

The distal end 20 of the tube 14 includes an expandable cage 24 configured to be expandable between a first cage position (FIG. 1) where the expandable cage 24 is collapsed and a second cage position (FIG. 2) where the expandable cage 24 is expanded. As shown in FIG. 2, the expandable cage 24 defines a substantially symmetrical zigzag shape when in the second cage position.

The expandable cage 24 can be fabricated from a biocompatible, shape memory alloy, such as nitinol. Additionally or alternatively, the expandable cage 24 can be fabricated from a material with a low thermal conductivity. It is known in the art that shape memory alloys can be configured to define one shape at a first physical state and another shape at a second physical state. Thus, in the non-limiting example where the expandable cage 24 is fabricated from a biocompatible, shape memory alloy, the physical state of the expandable cage 24 (e.g., apply a current, or alter the temperature) can be changed to expand the expandable cage 24 between the first cage position and the second cage position. Alternatively or additionally, the expandable cage 24 can be coated with a biocompatible, adhesive that adheres to human tissue.

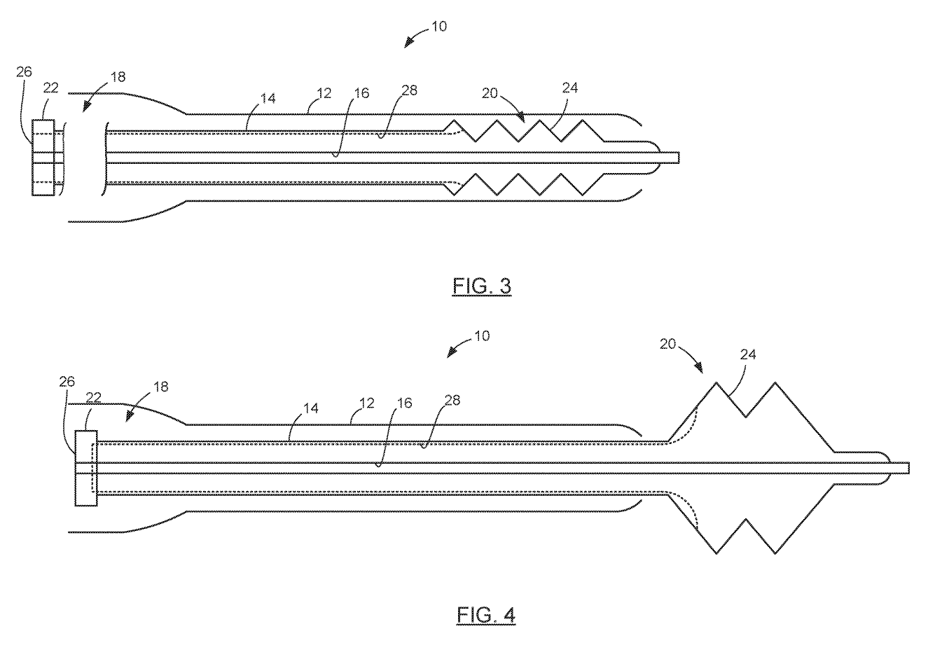

FIGS. 3 and 4 show another non-limiting example of the tissue displacement apparatus 10 where the actuation element 22 includes a push button 26 integrally formed within the actuation element 22. The push button 26 is configured to be actuated by a user of the tissue displacement apparatus 10. The push button 26 is coupled to a mechanical linkage 28 that is attached to the expandable cage 24. The push button 26 is configured to expand the expandable cage 24 between the first cage position (FIG. 3) and the second cage position (FIG. 4) when actuated.

In other non-limiting examples, the expandable cage 24 can be expanded between the first cage position and the second cage position using a spring-like mechanism, or the expandable cage 24 can be pre-stressed such that the expandable cage 24 automatically expands between the first cage position and the second cage position when the tube 14 is moved between the first tube position and the second tube position.

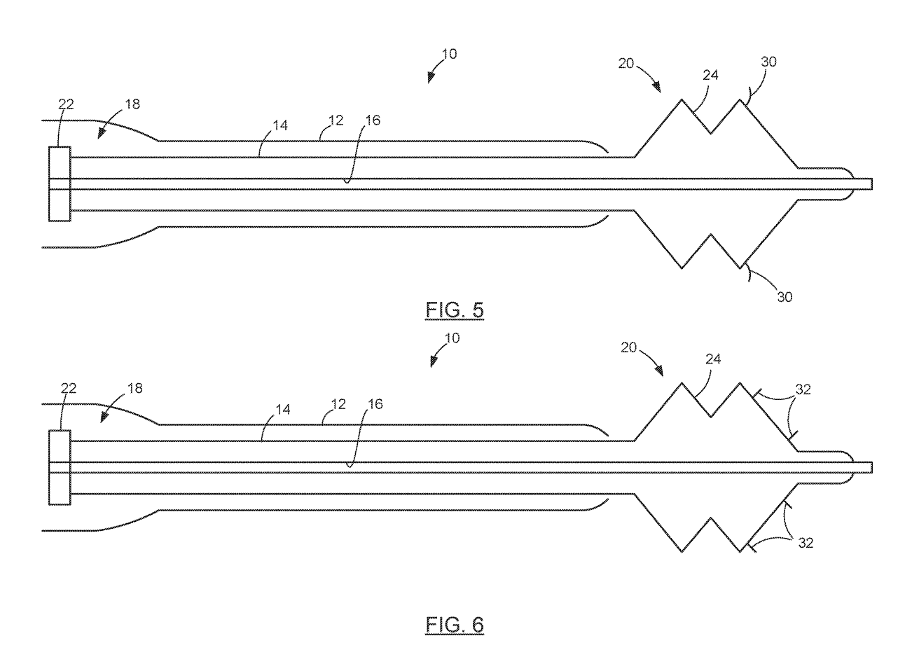

FIG. 5 shows one non-limiting example of the tissue displacement apparatus 10 where one or more anchors 30 are attached to the expandable cage 24. The anchors 30 are configured to anchor the expandable cage 24 when the expandable cage 24 is in the second cage position by providing resistance to the tube 14 moving between the first tube position and the second tube position. The illustrated anchors 30 are arranged on the expandable cage 24; however, in other non-limiting examples, the anchors 30 can be arranged at any location along the distal end 20 of the tube 14. As shown in FIG. 5, the tissue displacement apparatus 10 includes two anchors 30. In other non-limiting examples the tissue displacement apparatus 10 can include more or less than two anchors.

FIG. 6 shows another non-limiting example of the tissue displacement apparatus 10 where one or more temperature sensors 32 arranged on the distal end 20 of the tube 14. The temperatures sensors 32 are configured to measure the temperature of tissue surrounding or in contact with the expandable cage 24. The temperatures measured by the temperature sensors 32 can be communicated to a readout (not shown) for viewing by a user of the tissue displacement apparatus 10. As described above, during ablation procedures, the ablation probe generates extreme temperatures that can damage healthy tissue. The temperature sensors 32 can provide real time feedback of the temperature of healthy tissue surrounding or in contact with the expandable cage 24. Additionally, since the 3-D positions of the temperature sensors 32 with respect to one another can be geometrically calculated, the temperature data measured by the temperature sensors 32 can be input into a 3-D model of the temperature of the tissue surrounding or in contact with the expandable cage 24. The 3-D model can be displayed to the user via a display or user interface to aid in preventing damage to healthy tissue.

As shown in FIG. 6, the tissue displacement apparatus 10 includes four temperature sensors 32 arranged on opposing sides of the expandable cage 24 at two different axial locations along the expandable cage 24. By providing temperatures sensors 32 axially along the expandable cage 24, a profile of the temperature distribution of the tissue can be provided to a user of the tissue displacement apparatus 10. In other non-limiting examples, the tissue displacement apparatus 10 can include more or less than four temperature sensors 32 arranged at any location along the distal end 20 of the tube 14. The illustrated temperature sensors 32 are probe-type sensors that slightly protrude from the expandable cage 24. In another non-limiting example, the temperature sensors 32 can be integrated into, or within, the distal end 20 of tube 14.

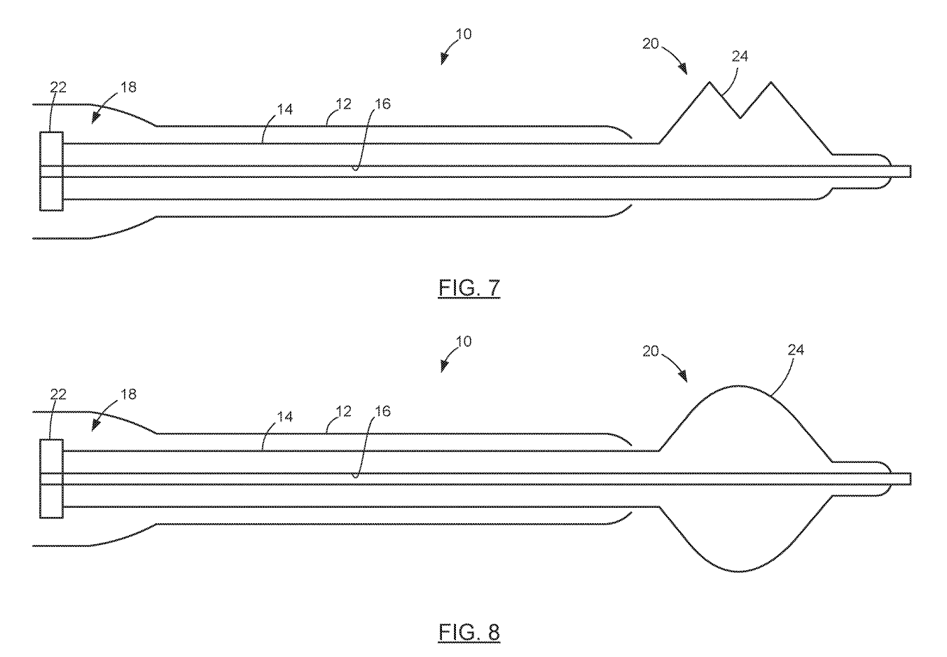

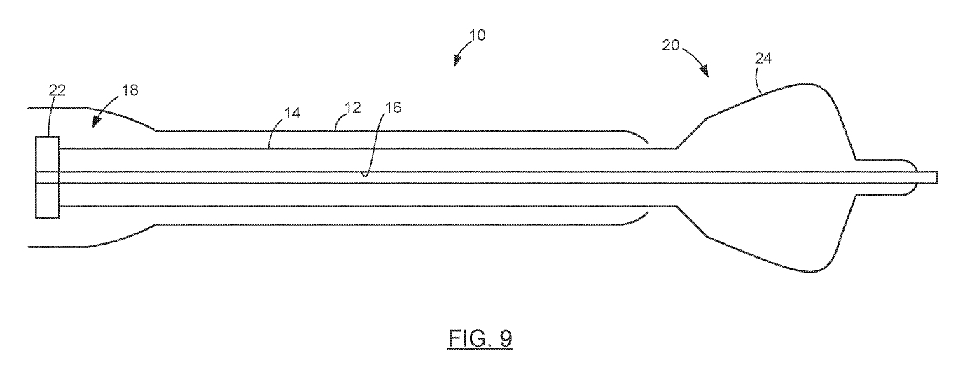

As described above and shown in FIGS. 2 and 4, the expandable cage 24 can define a substantially symmetric zigzag shape when in the second cage position. It should be known that this is but one non-limiting example, and the expandable cage 24 can define a different shape to conform to a specific region or position within a patient's body. For example, as shown in FIGS. 7, the expandable cage 24 can define a substantially asymmetrical shape when in the second cage position. In another non-limiting example, as shown in FIG. 8, the expandable cage 24 can define a substantially round shape when in the second cage position. In yet another non-limiting example, as shown in FIG. 9, the expandable cage 24 can define a substantially differential shape when in the second cage position. This is, the expandable cage 24 can expand a varying radial distance axially along the expandable cage 24. In other non-limiting examples, the expandable cage 24 can define an alternative shape to conform to specific cavities, pathways, or regions within a patient's body.

One non-limiting example of using the tissue displacement apparatus 10 during an ablation procedure performed within a patient's body will be described with reference to FIGS. 1-9. Typically, the user, or individual performing the ablation procedure, is a physician, or another trained medical professional. During the ablation procedure, the tissue displacement apparatus 10 within the external tube 12 is guided by a user, via medical imaging techniques (e.g., CT, MRI, X-Ray, etc.), to a location within the patent's body that includes aberrant tissue (e.g., tumor, atypical cells, etc.) or to a location within the patient's body that includes obstructive tissue in the path to the aberrant tissue. Alternatively or additionally, a contrast agent can be flown through the central lumen 16 of the tube 14 to enable the position of the distal end 20 of tissue displacement apparatus 10 within the patient more clearly visible using the medical imaging techniques.

Once the distal end 20 of the tube 14 is positioned in a desired location within the patient's body, the user can grip the actuation element 22 and move the tube 14 from the first tube position (FIGS. 1 and 3) to the second tube position (FIGS. 2 and 4) thereby protruding the distal end 20 of the tube 14 from the external tube 12. Once the tube 14 is in the second tube position, the expandable cage 24 can be expanded from the first cage position (FIGS. 1 and 3) to the second cage position (FIGS. 2 and 4). As described above, the expandable cage 24 can be fabricated from a shape memory alloy. In this non-limiting example, a current can be applied along the tube 14 to the expandable cage 24 to expand the expandable cage 24 to the second cage position. Alternatively, as shown in FIGS. 3 and 4, the user can actuate the push button 26 to enable the mechanical linkage 28 to expand the expandable cage 24 from the first cage position (FIG. 3) to the second cage position (FIG. 4). In still other non-limiting examples, the expandable cage 24 can be expanded between the first cage position and the second cage position using a spring-like mechanism, or the expandable cage 24 can be pre-stressed such that the expandable cage 24 automatically expands between the first cage position and the second cage position when the tube 14 is moved between the first tube position and the second tube position.

During the expansion of the expandable cage 24, the tissue surrounding the expandable cage 24 is displaced, or expanded outwardly, thereby providing the user a clear path to the aberrant tissue. The tissue surrounding the expandable cage 24 will remain displaced until the user instructs the expandable cage 24 to collapse to the first cage position. Thus, the tissue displacement apparatus 10 displaces the desired tissue while the user maintains use of both of their hands. Additionally, as described above and shown in FIG. 5, the tissue displacement apparatus 10 can include anchors 30, which, when the expandable cage 24 expands to the second cage position, can anchor in the surrounding tissue and prevent the tissue displacement apparatus 10 from displacing or shifting from its position within the patient. Alternatively or additionally, the expandable cage 24 can be coated in a biocompatible adhesive, as described above, which can aid in preventing the tissue displacement apparatus 10 from displacing or shifting from its position within the patient.

Once the expandable cage 24 is in the second cage position, an ablation probe (not shown) can then be inserted into a patient along the external tube 12 and through or along side the expandable cage 24 such that a tip of the ablation probe contacts the aberrant tissue. With tip of the ablation probe in contact with the aberrant tissue, the user can instruct the ablation probe to provide heat (e.g., radiofrequency ablation) or cold (e.g., cryoablation) to the aberrant tissue to ablate the aberrant tissue. As described above, the expandable cage 24 can be fabricated from a material with a low thermal conductivity. This can hinder the extreme temperatures generated by the ablation probe from transferring to the healthy tissue surrounding the expandable cage 24, thereby thermally isolating the healthy tissue. Additionally, while the ablation probe is performing the ablation of the aberrant tissue, the user can monitor the temperatures of the surrounding tissue measured by the temperature sensors 32, as shown in FIG. 6, to determine if healthy tissue surrounding the aberrant tissue is being damaged by the temperatures generated by the ablation probe. Once the user has ablated the desired amount of aberrant tissue, the user can collapse the expandable cage 24 from the second cage position to the first cage position and then the user can move the tube 14 from the second tube position to the second tube position and withdraw the tissue displacement apparatus 10 within the external tube 12 from the patient's body.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

We claim:

1. A tissue displacement apparatus configured to be slideably received within an external tube configured to be placed within a patient's body, the tissue displacement apparatus comprising:
a tube defining a central lumen and a distal end, the tube configured to be moveable between a first tube position where the distal end of the tube is arranged within the external tube and a second tube position where the distal end of the tube protrudes from the external tube;
an expandable cage arranged on the distal end of the tube and expandable between a first cage position where the expandable cage is collapsed and a second cage position where the expandable cage is expanded; and
wherein, when the tube is in the first tube position, the expandable cage is in the first cage position and wherein when the tube moves to the second tube position, the expandable cage expands to the second cage position.

2. The tissue displacement apparatus of claim 1, wherein the expandable cage is fabricated from a biocompatible shape-memory alloy.

3. The tissue displacement apparatus of claim 1, further comprising at least one anchor configured to anchor the expandable cage when the expandable cage is in the second cage position.

4. The tissue displacement apparatus of claim 1, wherein the expandable cage is coated with a biocompatible adhesive.

5. The tissue displacement apparatus of claim 1, further comprising at least one temperature sensor arranged on or integrated into the distal end of the tube.

6. The tissue displacement apparatus of claim 1, wherein the expandable cage is configured to define a symmetrical shape when the expandable cage is in the second cage position.

7. The tissue displacement apparatus of claim 1, wherein the expandable cage is configured to define an asymmetrical shape when the expandable cage is in the second cage position.

8. The tissue displacement apparatus of claim 1, wherein the expandable cage is configured to define a substantially round shape when the expandable cage is in the second cage position.

9. The tissue displacement apparatus of claim 1, wherein the expandable cage is configured to define a differential shape when the expandable cage is in the second cage position.

10. A tissue displacement apparatus configured to be slideably received within an external tube configured to be placed within a patient's body, the tissue displacement apparatus comprising:
a tube defining a central lumen, a distal end, and a proximal end, the tube configured to be moveable between a first tube position where the distal end of the tube is arranged within the external tube and a second tube position where the distal end of the tube protrudes from the external tube;
an expandable cage arranged on the distal end of the tube and expandable between a first cage position where the expandable cage is collapsed and a second cage position where the expandable cage is expanded; and
an actuation element arranged on the proximal end of the tube and including a push button, the push button configured to expand the expandable cage between the first cage position and the second cage position when actuated.

11. The tissue displacement apparatus of claim 10, wherein the push button is coupled to a mechanical linkage that is attached to the expandable cage.

12. The tissue displacement apparatus of claim 10, further comprising at least one anchor configured to anchor the expandable cage when the expandable cage is in the second cage position.

13. The tissue displacement apparatus of claim 10, wherein the expandable cage is coated with a biocompatible adhesive.

14. The tissue displacement apparatus of claim 10, further comprising at least one temperature sensor arranged on or integrated into the distal end of the tube.

15. The tissue displacement apparatus of claim 10, wherein the expandable cage is configured to define a symmetrical shape when the expandable cage is in the second cage position.

16. The tissue displacement apparatus of claim 10, wherein the expandable cage is configured to define an asymmetrical shape when the expandable cage is in the second cage position.

17. The tissue displacement apparatus of claim 10, wherein the expandable cage is configured to define a substantially round shape when the expandable cage is in the second cage position.

18. The tissue displacement apparatus of claim 10, wherein the expandable cage is configured to define a differential shape when the expandable cage is in the second cage position.
